(19) United States
(12) Patent Application Publication
(10) Pub. No.: US 2026/0083772 A1
(43) Pub. Date: Mar. 26, 2026

(54) POWDER COMPOSITIONS COMPRISING FIBER

(71) Applicant: Kenvue Brands LLC, Summit, NJ (US)

(72) Inventors: Katherine C. Dectis, Langhorne, PA (US); Nancy Edge, Summit, NJ (US)

(21) Appl. No.: 19/338,271

(22) Filed: Sep. 24, 2025

Related U.S. Application Data
(60) Provisional application No. 63/699,350, filed on Sep. 26, 2024.

(51) Int. Cl. A61K 31/736; A61K 9/14; A61K 36/48; A61P 1/14

(52) U.S. Cl. CPC A61K 31/736; A61K 9/14; A61K 36/48; A61P 1/14

(57) ABSTRACT

Powder compositions comprising one or more therapeutic ingredients including fiber and processes of manufacturing the same. Such compositions can be used as a prebiotic dietary supplement to maintain and/or improve the gut microbiome and overall gut health of a human.

### CROSS-REFERENCE TO RELATED APPLICATIONS

This application claims the benefit of U.S. provisional application 63/699,350 filed on Sep. 26, 2024, the complete disclosure of which is hereby incorporated herein by reference for all purposes.

### 1. FIELD

The present disclosure relates to powder compositions, and in particular powder compositions comprising one or more therapeutic ingredients including fiber, and processes of manufacturing the same. The present disclosure further relates to the use of such powder compositions as a prebiotic dietary supplement to maintain and/or improve the gut microbiome and overall gut health of a human.

### 2. BACKGROUND

The human gut microbiome has a role in overall health in influencing digestion, metabolism, and immune function. Prebiotics are among the various components that support gut health, for example, by fostering growth or activity of beneficial microorganisms such as certain bacteria and fungi while suppressing the growth of pathogenic bacteria. Prebiotics include inulin, fructooligosaccharides (FOS), galactooligosaccharides (GOS), and resistant starch. Despite the recognized benefits of prebiotics, there are several challenges associated with their use. For example, the stability and bioavailability of prebiotics in various dietary supplements remain important factors for effective formulations. There remains a continued need in the art for novel prebiotic formulations that enhance stability, improve bioavailability, and maximize the beneficial effects of prebiotics on the gut microbiota. The present disclosure addresses these and other needs.

### 3. SUMMARY

The present disclosure provides a powder composition including a first therapeutic ingredient and a second therapeutic ingredient. The first and second therapeutic ingredients include fiber and are present in a ratio of about 3:1.

In certain embodiments, the first therapeutic ingredient can be Partially Hydrolyzed Guar Gum (PHGG).

In certain embodiments, the second therapeutic ingredient can be gum acacia.

In certain embodiments, the powder composition can consist essentially of the first and second therapeutic ingredients.

The present disclosure further provides a method of maintaining or improving the gut microbiome and overall gut health of a human. The method includes administering the powder composition of the present disclosure to the human.

### 4. DETAILED DESCRIPTION

The presently disclosed subject matter relates to powder compositions comprising one or more therapeutic ingredients including fiber and methods of manufacturing the same. Such compositions can be used as a prebiotic dietary supplement, for example, to maintain and/or improve the gut microbiome and overall gut health of a human.

These and other aspects of the disclosed subject matter are discussed in more detail below and in the Examples. For clarity, and not by way of limitation, this detailed description is divided into the following sub-portions:
4.1. Definitions;
4.2. Powder Compositions;
4.3. Methods of Making Powder Compositions;
4.4. Methods of Using Powder Compositions; and
4.5. Features of Powder Compositions.

### 4.1. Definitions

The terms used in this specification generally have their ordinary meanings in the art within the context of this disclosure and in specific context where each term is used. Certain terms are discussed below, or elsewhere in the specification, to provide additional guidance in describing the compositions and methods of the disclosure and how to make and use them.

As used in the specification and the appended claims, the singular forms “a,” “an,” and “the” include the plural referents unless the context clearly dictates otherwise. Thus, for example, reference to “an ingredient” includes mixtures of ingredients.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. The general convention in the scientific and technical literature is applied: the last decimal place of a numerical value indicates its degree of accuracy. Where no other error margins are given, the maximum margin is ascertained by applying the rounding-off convention to the last decimal place, for example for a measurement of 3.5%, the error margin is 3.45-3.54.

As used herein, “comprises,” “comprising,” or any other variation thereof, are intended to cover a non-exclusive inclusion, such that a process, method, article, or apparatus that comprises a list of elements does not include only those elements but can include other elements not expressly listed or inherent to such process, method, article, or apparatus.

The expression “one or more” is synonymous with “at least one” and includes individual components as well as mixtures/combinations.

All percentages, parts and ratios herein are based upon the total weight of the cleaning compositions of the present disclosure, unless otherwise indicated.

As used herein, the terms “% w/w” or “weight percent” refers to the percentage of an ingredient(s)/the total percentage by weight of the composition (100%). The terms “% w/w” or “weight percent” refer to the quantity by weight of a constituent or component. The terms “weight percent”, “wt-%”, “wt. %”, and “wt %” are used interchangeably.

All publications and patent applications cited in this specification are herein incorporated by reference, and for any and all purposes, as if each individual publication or patent application were specifically and individually indicated to be incorporated by reference. The present disclosure controls if there an inconsistency between the present disclosure and any incorporated publications or patents.

### 4.2. Powder Compositions

Powder compositions of the present disclosure include one or more therapeutic ingredients including fiber. In certain embodiments, the powder composition can include at least one therapeutic ingredient including fiber. In particular embodiments, the powder composition can include at least two therapeutic ingredients (e.g., a first therapeutic ingredient and a second therapeutic ingredient) each including fiber. For example, and not by way of limitation, the powder composition can consist of two therapeutic ingredients each including fiber. The one or more therapeutic ingredients including fiber can include Partially Hydrolyzed Guar Gum (PHGG), gum acacia, or combinations thereof.

In certain aspects, the powder composition can include a first therapeutic ingredient and a second therapeutic ingredient. The first and second therapeutic ingredients can each include fiber. The first therapeutic ingredient can be present in an amount of from about 25% to about 90%, about 30% to about 80%, or about 50% to about 75% by weight, based on the total weight of the powder composition. The second therapeutic ingredient can be present in an amount of from about 1% to about 40%, about 10% to about 30%, or about 15% to about 25% by weight, based on the total weight of the powder composition. The first therapeutic ingredient and the second therapeutic ingredient can be present in a ratio of about 3:1. In particular embodiments, the first therapeutic agent can be present in an amount of about 75% by weight, based on the total weight of the powder composition and the second therapeutic agent can be present in an amount of about 25% by weight, based on the total weight of the powder composition. In certain embodiments, the first therapeutic ingredient can include Partially Hydrolyzed Guar Gum (PHGG), and the second therapeutic ingredient can include gum acacia.

### 4.3. Methods of Making Powder Compositions

In certain embodiments, a method of manufacturing a powder composition of the present disclosure is provided. A wide variety of processes can be used to provide the powder composition of the present disclosure. In certain embodiments, the powder composition can be produced by adding the one or more therapeutic ingredients in certain proportions into a suitable blender (e.g., a V-blender) and blended. The mixture can be blended NLT for at least 10 minutes, at least 15 minutes, at least 20 minutes, or at least 25 minutes. In certain embodiments, the mixture can be blended NLT for about 10 minutes. The blended mixture can be placed into a suitable container, e.g., barrels. The blended mixture can be dispensed (e.g., from the suitable container or barrels) into suitable packaging such as separate packaging in the form of sachets, stick packs, or cannisters. In certain embodiments, the blended mixture can be dispensed or filled into suitable packaging by hand or mechanically.

### 4.4. Methods of Using Powder Compositions

Powder compositions of the present disclosure can be used, for example, alone or in combination with food or drink. In certain aspects, the powder composition can be reconstituted with water, juice, milk, apple sauce, yogurt, or other foods (e.g., 7.5 g of powder composition in a food or beverage of choice). The powder compositions of the present disclosure can be administered orally. Methods of using the powder compositions as a prebiotic dietary supplement to maintain and/or improve the gut microbiome and overall gut health of a human are provided.

### 4.5. Features of Powder Compositions

The present disclosure provides for powder compositions. Such powder compositions can be off white to light yellow in appearance.

### 5. EXAMPLES

The following Examples are intended to illustrate, but not to limit, the subject matter in any manner, shape, or form, either explicitly or implicitly.

### Example 1: Fiber Powder Blend

The present example was prepared using the formula in Table 1.

TABLE 1
Fiber Powder Formulation

| | | Actual | Raw | |
|---|---|---|---|---|
| | | Fiber | material | |
| Ingredient | Function | mg/Serving³ | mg/Serving³ | % W/W |
| Partially Hydrolyzed Guar | Therapeutic | 4632 | 5600 | 74.67 |
| Gum (PHGG)¹ | Ingredient (Fiber) | | | |
| Gum Acacia² | Therapeutic | 1368 | 1900 | 25.33 |
| | Ingredient (Fiber) | | | |
| TOTAL Serving Size | | | 7500 | 100.0 |

¹Commercially available as Sunfiber ® from the Taiyo International Corporation, also known as Partially hydrolyzed guar gum (PHGG)
²Commercially available as Inavea ™ Essential W from the Nexira Corporation
³The actual Fiber mg/Serving is converted from the mg/Serving based on a potency of 85% for Sunfiber ® (with 2.8% overage) and 72% for Inavea ™

### Processing Directions:

1. The Partially Hydrolyzed Guar Gum and Guma Acacia were added to a suitable V-Blender and blended end-over-end for at least 10 minutes.
2. The blended mixture is dispensed into barrels.
3. From the barrels, the blended mixture is filled into separate packages as sachets, stick packs, or cannisters.

### Example 2: In-Vitro Gastrointestinal Analysis

Individual fiber components and various ratios of fiber compositions were tested to determine behavior within environments that mimic human gastrointestinal environments. These were analyzed using the Simulator of Human Intestinal Microbial System (SHIME® model) as published in Nutraceuticals 2023, 3, 489-498. https://doi.org/10.3390/nutraceuticals3040035. The model determined the production of short chain fatty acids and microbial populations.

Part A: Study Overview. The colonic microbiome of 6 healthy children (3 children between 15 months and 30 months of age and 3 additional healthy children between 31 months and 12 years of age) were simulated using the full SHIME® (proximal and distal colonic bioreactor) and the impact of 3 weeks dosing of the test products were assessed and compared to a negative control. By testing 3 donors of each age group, it was expected to cover the evolution in the children microbiome composition in function of age. Scientific publications have defined three phases in the microbiome development of infants and children: (i) a developmental phase between 3 and 14 months of age (which is not in scope in this Example), (ii) a transitional phase between 15 and 30 months of age, and (iii) a stable phase from 31 months onwards.

Part B: Methodology and Experimental Setup. The typical reactor setup of the SHIME®, representing the gastrointestinal tract of the adult human, was described by Molly, K., et al. (1993). Development of a 5-step multichamber reactor as a simulation of the human intestinal microbial ecosystem. Applied Microbiology and Biotechnology 39 (2): 254-258. It consists of a succession of five reactors simulating the different parts of the human gastrointestinal tract. The first two reactors are of the fill-and-draw principle to simulate different steps in food uptake and digestion, with peristaltic pumps adding a defined amount of SHIME® feed (140 mL 3×/day) and pancreatic and bile liquid (60 mL 3×/day), respectively to the stomach (VI) and small intestine (V2) compartment and emptying the respective reactors after specified intervals. The last three compartments simulate the large intestine. These reactors are continuously stirred, they have a constant volume and pH control. Retention time and pH of the different vessels are chosen to resemble in vivo conditions in the different parts of the colon. Upon inoculation with fecal microbiota, these reactors simulate the ascending (V3), transverse (V4) and descending (V5) colon. Inoculum preparation, retention time, pH, temperature settings and reactor feed composition were previously described by Possemiers S. et al. (2004). PCR-DGGE-based quantification of stability of the microbial community in a simulator of the human intestinal microbial ecosystem. FEMS Microbiol Ecol. 49 (3): 495-507. Upon stabilization of the microbial community in the different regions of the colon, a representative microbial community is established in the three colon compartments, which differs both in composition and functionality in the different colon regions.

A typical long-term SHIMER experiment consists of three distinct phases: (1) A stabilization phase of up to 3 weeks, during which the microbial community differentiates to the specific environmental conditions of the reactors. (2) A control period, during which the baseline properties of the microbial community can be sampled during 6 consecutive weekdays (excluding weekend). (3) The treatment phase, during which a treatment is applied to the microbial communities and its effects are observed. Usually, this phase comprises 3 weeks.

The normal repeated administration SHIME® setup was adapted to the shortened form, in which no stabilization and control phase are included. Instead, the additional negative control arm was incorporated for each donor to function as a reference. The typical long-term SHIME® setup was therefore adapted to a short-term SHIME® setup, which is divided in the following stages:

Inoculation period: On day 0, the colon reactors were inoculated with an appropriate fecal sample and were allowed to grow and colonize the reactor. After this overnight incubation, the colon reactors were fed with the basic nutritional matrix for two more days, to support the maximum diversity of the gut microbiota originally present in the fecal inoculum. This also allows the microbial community to differentiate in the different reactors depending on the local environmental conditions.

Treatment period (TR): During this 3-week period, the SHIME® reactor was operated under nominal conditions and fed 3×/day with the standard SHIME® nutritional medium. In the treatment arms, this diet was supplemented with the test product (i.e., the formulation in Example 1, Table 1). Samples were taken from the colon reactors throughout this period to investigate the specific effect on the resident microbial community composition and activity. Other fiber types were tested including Pure Gum Acacia; PHGG; PHGG:Gum Acacia (50:50); and Gum Acacia (Alternate). The Pure Gum Acacia and PHGG were the same ingredients utilized in Example 1, Table 1 and for the PHGG:Gum Acacia (50:50) product. An alternate Gum Acacia, Pre-Hydrated® Gum Arabic FT (ARAB PH-FT) commercially available from Ingredion was also tested.

During each period, the model was fed 3×/day with the SHIME® nutritional medium (17.2 g/L PDNM005 for toddlers and 15.6 g/L PDNM001B for young children; set at pH=3.0 (toddlers) or pH=2.0 (young children)) and pancreatic juice (NaHCO₃, oxgall and pancreatin).

The three feeding cycles of the SHIMER for this Example started at 9 h, 17 h and 1 h (SHIME® time). The feed in each cycle entered in the colon 3 h after the start of the cycle (so at 12 h, 20 h and 4 h SHIME® time).

Following samples were collected from the SHIME® experiment:
1. Overall fermentative activity:
   a. Acid/base consumption
2. Microbial Community Activity
   a. Short Chain Fatty Acids (SCFA): Acetate, Butyrate and Propionate
   b. Lactate
   c. Ammonium
3. Microbial Community Composition
   a. llumina 16S rRNA sequencing
   b. Quantification of total bacterial cells by flow cytometry

Part C: Results. The results of the study are provided in Table 2.

TABLE 2
Study Results

| | Proximal Colon (PC) | | Distal Colon (DC) | |
|---|---|---|---|---|
| Fiber Type | Metabolic Shift | Microbial Shift | Metabolic Shift | Microbial Shift |
| Pure Gum | Acetate increase | Subdoligranulum | Acetate increase | Subdoligranulum |
| Acacia¹ | Butyrate increase | increase | Butyrate increase | increase |
| | | | Propionate increase | |
| | | | Ammonium decrease | |
| PHGG² | Acetate increase | Roseburia increase | Acetate increase | Roseburia increase |
| | Butyrate increase | Bifidobacterium | Butyrate increase | Bifidobacterium |
| | Propionate increase | increase | Propionate increase | increase |
| | Ammonuim decrease | | Ammonium decrease | |
| PHGG:Gum | Acetate increase | Subdoligranulum | Acetate increase | Subdoligranulum |
| Acacia (75:25) | Butyrate increase | increase | Butyrate increase | increase |
| (Example 1, | Propionate decrease | Roseburia increase | Ammonium decrease | Roseburia increase |
| Table 1) | Ammonium decrease | Bifidobacterium | | Bifidobacterium |
| | | increase | | increase |
| PHGG:Gum | Acetate increase | Subdoligranulum | Acetate increase | Subdoligranulum |
| Acacia (50:50) | Butyrate increase | increase | Butyrate increase | increase |
| | Ammonium decrease | Roseburia increase | Propionate increase | Roseburia increase |
| | | Bifidobacterium | Ammonium decrease | Bifidobacterium |
| | | increase | | increase |
| Gum Acacia³ | Acetate increase | | Acetate increase | Subdoligranulum |
| (Alternate) | Butyrate increase | | Ammonium decrease | increase |
| | Propionate decrease | | | |
| | Ammonium decrease | | | |

¹Commercially available as Inavea ™ from the Nexira Corporation
²Partially Hydrolyzed Guar Gum: Commercially available as Sunfiber ® from the Taiyo International Corporation
³Commercially available as Pre-Hydrated ® Gum Arabic FT (ARAB PH-FT) from Ingredion Corporation

Results Summary: Fermentative activity can be categorized into saccharolytic fermentation and proteolytic fermentation types. Saccharolytic fermentation includes the production of short chain fatty acids such as acetate, propionate and butyrate. Saccharolytic fermentation also includes the production of lactate. Proteolytic fermentation includes the production of branched chain fatty acids and ammonium.

1. A powder composition comprising:
a first therapeutic ingredient; and
a second therapeutic ingredient,
wherein the first and second therapeutic ingredients comprise fiber, and
wherein the first and second therapeutic ingredients are present in a ratio of about 3:1.

2. The powder composition of claim 1, wherein the first therapeutic ingredient is Partially Hydrolyzed Guar Gum (PHGG).

3. The powder composition of claim 1, wherein the second therapeutic ingredient is gum acacia.

4. The powder composition of claim 1, wherein the powder composition consists essentially of the first and second therapeutic ingredients.

5. A method of maintaining or improving the gut microbiome and overall gut health of a human, comprising administering the powder composition of claim 1 to the human.
